(19) United States
(12) Patent Application Publication
(10) Pub. No.: US 2014/0155630 A1
(43) Pub. Date: Jun. 5, 2014

(54) Processes for the Preparation of Enamines

(71) Applicants: Douglas C. Bland, Midland, MI (US); Todd W. Toyzan, Freeland, MI (US)

(72) Inventors: Douglas C. Bland, Midland, MI (US); Todd W. Toyzan, Freeland, MI (US)

(73) Assignee: Dow AgroSciences LLC, Indianapolis, IN (US)

(21) Appl. No.: 14/172,580

(22) Filed: Feb. 4, 2014

Related U.S. Application Data
(63) Continuation of application No. 13/303,187, filed on Nov. 23, 2011, now abandoned.
(60) Provisional application No. 61/419,296, filed on Dec. 3, 2010.

(51) Int. Cl. C07D 295/084

(52) U.S. Cl. CPC C07D 295/084
USPC 548/574

(57) ABSTRACT

The invention disclosed in this document is related to the field of processes for the preparation of enamines

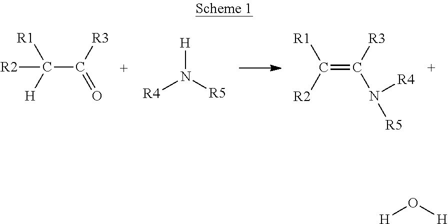

wherein R1, R2, R3, R4, R5, and further information are disclosed herein.

### CROSS-REFERENCE TO RELATED APPLICATIONS

This Application is a continuation of U.S. non-provisional application Ser. No. 13/303,187, which was filed on 23 Nov. 2011, and claims priority from, and benefit of U.S. provisional application 61/419,296, filed on Dec. 3, 2010. The entire contents of these application are hereby incorporated by reference into this Application.

### FIELD OF THE INVENTION

The invention disclosed in this document is related to the field of processes for the preparation of enamines.

### BACKGROUND OF THE INVENTION

Enamines are very useful molecules. They have been used in a wide variety of reactions such as, for example, electrophilic substitution and addition, oxidation and reduction, and cycloaddition (J. Kang, Y. R. Cho, and J. H. Lee, Bull. Korean Chem Soc. Vol. 13, No. 2, 1992).

An early method for preparing enamines involved the condensation of aldehydes and ketones with secondary amines (C. Mannich and H. Davidsen, Ber., 69, 2106 (1936)). Mannich and Davidsen discovered that the condensation reaction of an aldehyde with a secondary amine could be conducted at temperatures near 0° C. in the presence of potassium carbonate (K₂CO₃), but however, the condensation reaction of a ketone with a secondary amine required calcium oxide (CaO) and elevated temperatures. Later, Herr and Heyl discovered that this type of condensation reaction could be improved by removing water (H₂O) during an azeotropic distillation with benzene (M. E. Herr and F. W. Heyl, J. Am. Chem. Soc., 74, 3627 (1952); F. W. Heyl and M. E. Herr, J. Am. Chem. Soc., 75, 1918 (1953); M. E. Herr and F. W. Heyl, J. Am. Chem. Soc., 75, 5927 (1953); F. W. Heyl and M. E. Herr, J. Am. Chem. Soc., 77, 488 (1955)). Since these publications a number of modifications have been disclosed. Usually, these modifications are based on using dehydration reagents such as K₂CO₃, CaO, p-toluenesulfonic acid (TsOH), boron trifluoride diethyl etherate (BF₃-OEt₂), acetic acid (AcOH), magnesium sulfate (MgSO₄), calcium hydride (CaH₂), titanium tetrachloride (TiCl₄), and molecular sieves (see J. Kang above). Other modifications deal with chemically converting water to something else during the condensation reaction (see J. Kang above). An extensive summary of the vast number of methods to prepare enamines is discussed in “ENAMINES, Synthesis, Structure, and Reactions, 2^{nd} Edition, Edited by A. G. Cook, Chap. 2, (1988). Specific examples of processes to prepare enamines can be found in the following:

U.S. Pat. No. 3,074,940 which discloses that certain aldehydes form azeotropes with water which can be used to remove the reaction water formed during certain enamine condensation reactions;

U.S. Pat. No. 3,530,120 which discloses conducting certain enamine condensation reactions in an inert atmosphere with certain arsine molecules;

U.S. Pat. No. 5,247,091 which discloses conducting certain enamine condensation reactions in an aqueous media;

S. Kaiser, S. P. Smidt, and A. Pfaltz, Angew. Int. Ed. 2006, 45, 5194-5197—See Supporting information pages 10-11; and

WO 2009/007460 A2, see page 13, example 1.a.

Enamines such as 1-(3-thiobut-1-enyl)pyrrolidine are useful intermediates for the preparation of certain new insecticides (see, for example, U.S. Patent Publications 2005/0228027 and 2007/0203191). Current known processes to make such thioenamines are not efficient in producing such enamines due to a variety of reasons—there are problems in preventing thermal degradation of the thioenamine, and while using potassium carbonate is an effective desiccant, it is problematic to filter such desiccant during larger than lab-scale production. Thus, a process is needed to remove water during these types of condensation reactions without using solid desiccants, or using temperature conditions that promote the thermal degradation of such enamines.

### DETAILED DESCRIPTION OF THE INVENTION

In general, the processes disclosed in this document can be illustrated as in Scheme 1.

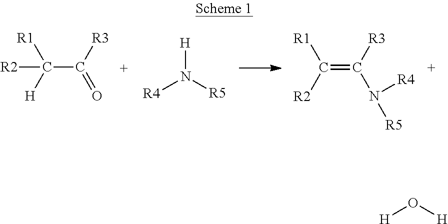

In general, the invention is a process comprising:

(A) contacting, in a reaction zone, a first mixture with a second mixture
(1) wherein said first mixture comprises a carbonyl (i.e. an aldehyde or a ketone) having the following formula

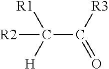

(a) wherein R1 and R2 is each independently selected from C₁-C₈ alkyl, C₃-C₈ cycloalkyl, C₂-C₈ alkoxyalkyl, C₇-C₁₂ arylalkyl, C₂-C₈ alkylaminoalkyl, aryl, and heteroaryl, each of which is independently substituted with one or more S-R6 wherein each R6 is independently selected from C₁-C₈ alkyl, C₃-C₈ cycloalkyl, C₂-C₈ alkoxyalkyl, C₇-C₁₂ arylalkyl, C₂-C₈ alkylaminoalkyl, aryl, and heteroaryl, and
(b) wherein R3 is selected from H, C₁-C₈ alkyl, C₃-C₈ cycloalkyl, C₂-C₈ alkoxyalkyl, C₇-C₁₂ arylalkyl, C₂-C₈ alkylaminoalkyl, aryl, and heteroaryl, and
(2) wherein said second mixture comprises a non-polar-high-boiling-point solvent and an amine having the following formula

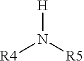

wherein R4 and R5 are each independently selected from C₁-C₈ alkyl, C₃-C₈ cycloalkyl, C₂-C₈ alkoxyalkyl, C₇-C₁₂ arylalkyl, C₂-C₈ alkylaminoalkyl, aryl, and heteroaryl, or R4 and R5 taken together with N represent a 5- or 6-membered saturated or unsaturated ring;

(B) reacting in said reaction zone said amine and said carbonyl to produce an enamine and H₂O, wherein reacting is conducted under distillation conditions comprising
(1) a pressure from about 100 Pascals (Pa) to about 120,000 Pa, and
(2) a temperature below about, but preferably below, the thermal decomposition temperature of said enamine during said reacting; and

(C) removing a vapor phase comprising said non-polar-high-boiling-point-solvent, amine, and H₂O; and

(D) condensing said vapor phase from step (C) to produce a condensate; and

(E) contacting said condensate from step (D) with a recovery mixture comprising H₂O and an amine-rejecting agent to produce a separate mixture comprising said amine; and

(F) optionally, returning said amine from step (E) back to said reaction zone.

Approximately equimolar quantities of said amine and said carbonyl can be used in the process, although excesses of one or the other may be employed. The molar ratio of amine to carbonyl can be from about 0.9 to about 1.2, however, a slight molar excess of amine to carbonyl is preferred, such as, for example, a molar ratio greater than 1 but less than about 1.1.

The reaction is conducted in the presence of a non-polar-high-boiling-point-solvent such as, hydrocarbon solvents, most preferably aromatic hydrocarbon solvents such as, for example, benzene, toluene, or xylene. Currently, toluene is a preferred solvent.

In another embodiment of this invention said reacting is conducted under distillation conditions comprising a pressure from about 1000 Pa to about 60,000 Pa and a temperature from about 10° C. to about 80° C.

In another embodiment of this invention said reacting is conducted under distillation conditions comprising a pressure from about 2500 Pa to about 30,000 Pa and a temperature from about 20° C. to about 70° C.

In another embodiment of this invention said reacting is conducted under distillation conditions comprising a pressure from about 5000 Pa to about 15,000 Pa and a temperature from about 25° C. to about 65° C. In another embodiment of this invention when producing 1-(3-methylsulfanyl-but-1-enyl)-pyrrolidine a temperature below about the thermal decomposition temperature of 1-(3-methylsulfanyl-but-1-enyl)-pyrrolidine during said reacting is preferred.

It is preferred in such processes that the condensation reaction be conducted under azeotropic conditions so that as much water can be removed as desired. It is also preferred if no desiccants be used to remove water.

In another embodiment of this invention, R1 and R2 are independently C₁-C₈ alkyl, C₃-C₈ cycloalkyl, each of which is independently substituted with one or more S-R6 wherein each R6 is independently selected from C₁-C₈ alkyl.

In another embodiment of this invention, R3 is H.

In another embodiment of this invention, R4 and R5 are each independently selected from C₁-C₈ alkyl, and C₃-C₈ cycloalkyl. In another embodiment of this invention R4 and R5 taken together with N represent a 5- or 6-membered saturated or unsaturated ring.

In another embodiment of this invention, said first mixture comprises pyrrolidine and said second mixture comprises 3-methylsulfanyl-butyraldehyde. In another embodiment of this invention, said enamine is 1-(3-methylsulfanyl-but-1-enyl)-pyrrolidine.

In another embodiment of this invention, the first mixture and second mixture can be contacted in the reaction zone simultaneously as they are added.

In another embodiment of this invention, said recovery mixture comprises an amine rejecting agent. An amine rejecting agent is an agent that is ionic and that dissolves in water readily, such as, for example, sodium hydroxide and brine solutions. Preferably the amine rejecting agent is concentrated in H₂O to greater than 25 weight percent sodium hydroxide, such as about 25 to about 50 weight percent sodium hydroxide.

### EXAMPLES

The examples are for illustration purposes and are not to be construed as limiting the invention disclosed in this document to only the embodiments disclosed in these examples.

### Comparative Example Preparation of 1-(3-methylthiobut-1-enyl)pyrrolidine.

A three-neck 250 mL round bottom flask equipped with a short path distillation head was connected to a receiver flask containing a dry-ice acetone condenser. To this reaction vessel was charged 19.8 g (0.28 mol) of pyrrolidine followed by 70 mL of toluene. The mixture was cooled in an ice-water bath until the internal reaction pot temperature was about 3° C. Then vacuum (about 3300 Pa) was applied to the system and then 94.4 g (0.14 mol) of 3-methylthiobutanal as a 17.5 wt % solution in toluene was continuously added to the reaction mixture via syringe over a one hour (h) period. The internal reaction temperature rose from 3° C. up to 18° C. during addition of the aldehyde solution. Distillate was also collected during aldehyde addition. Upon completing addition of the 3-methylthiobutanal solution, the distillation was continued for an additional 50 minutes (min) until the internal pot temperature reached 26° C. At this time, the vacuum was adjusted to about 2400 Pa and the distillation was continued for an additional 2.0 min until the internal pot temperature reached 24° C. The distillation was stopped and the reaction vessel was padded with nitrogen. The reactive distillation bottoms were isolated to give 74.91 g of 1-(3-methylthiobut-1-enyl)pyrrolidine was a 28 wt % yellow solution in toluene. Proton (¹H) NMR spectroscopic assay of the solution mixture (using benzyl acetate as the internal standard) indicated a 84% in-pot yield.

### Example #1 Preparation of 1-(3-methylthiobut-1-enyl)pyrrolidine.

A three-neck 250 mL round bottom flask was equipped with a Dean-Stark trap, addition funnel, and magnetic stir bar. On top of the Dean Stark trap was stacked a water cooled condenser followed by a dry-ice acetone condenser. To the Dean-Stark trap collection reservoir was charged 11 g of 50 wt % aqueous sodium hydroxide and this collection reservoir was cooled in an ice-water bath. To the 250 mL reaction vessel was charged 10.95 g (0.15 mol) of pyrrolidine followed by 70 mL of toluene. A vacuum (about 6600 Pa) was applied to the system and toluene was allowed to collect into the Dean-Stark trap collection reservoir. Once the reflux return from the Dean Stark trap to the reaction pot had been established, a 94.4 g (0.14 mol) of 3-methylthiobutanal as a 17.5 wt % in toluene solution was continuously added through the addition funnel over a 1 h and 15 min period. The internal reaction temperature was maintained below 24° C. during the aldehyde addition. Upon completing addition of the 3-methylthiobutanal, the distillation was stopped and the Dean-Stark trap reservoir was drained. The Dean-Stark trap reservoir was then filled with 2 mL of distilled water and the distillation was continued at about a 6600 Pa vacuum for 70 min until the internal pot temperature reached 30° C. At this time, the distillation was halted and the Dean-Stark trap reservoir was drained. The Dean-Stark trap was then replaced with a short path distillation head and the distillation was continued at about 6600 Pa for an additional 30 min until the pot temperature reached 33° C. The vacuum was adjusted to about a 2400 Pa and the distillation was continued until the pot temperature reached 21° C. at which time the distillation was halted and the reaction vessel was padded with nitrogen. A total of 59 g of distillate was collected overhead. The reactive distillation bottoms were isolated to give 72.26 g of 1-(3-methylthiobut-1-enyl)pyrrolidine was a 27.6 wt % yellow solution in toluene. Proton NMR spectroscopic assay of the solution mixture (using benzyl acetate as the internal standard) indicated a 83% in-pot yield.

In the comparative example about twice as much amine had to be used to obtain good yields as opposed to Example 1.

What is claimed is:

1. A process to produce 1-(3-methylsulfanyl-but-1-enyl)-pyrrolidine said process comprising:
(A) contacting, in a reaction zone, a first mixture with a second mixture
(1) wherein said first mixture comprises a carbonyl wherein said carbonyl is 3-methylsulfanyl-butyraldehyde, and
(2) wherein said second mixture comprises toluene and an amine wherein said amine is pyrrolidine;
(B) reacting in said reaction zone said amine and said carbonyl to produce an enamine and H₂O, wherein said enamine is 1-(3-methylsulfanyl-but-1-enyl)-pyrrolidine, and wherein reacting is conducted under azeotropic distillation conditions comprising
(1) a pressure from about 100 Pascals (Pa) to about 120,000 Pa, and
(2) a temperature below about the thermal decomposition temperature of said enamine during said reacting; and
(C) removing a vapor phase comprising said toluene, amine, and H₂O; and
(D) condensing said vapor phase from step (C) to produce a condensate; and
(E) contacting said condensate from step (D) with a recovery mixture comprising H₂O, toluene, amine, and sodium hydroxide to produce a separate mixture comprising said amine and said toluene, and another mixture comprising H2O and sodium hydroxide; and
(F) optionally, returning said mixture comprising amine and toluene from step (E) back to said reaction zone;
wherein said process no desiccants are used to remove water and
wherein said process the molar ratio of amine to carbonyl is greater than 1 but less than about 1.1.
